(19) United States
(12) Patent Application Publication
(10) Pub. No.: US 2006/0069040 A1
(43) Pub. Date: Mar. 30, 2006

(54) GHB compositions

(76) Inventor: Mortimer Mamelak, Toronto (CA)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 11/221,403

(22) Filed: Sep. 7, 2005

Related U.S. Application Data
(60) Provisional application No. 60/607,651, filed on Sep. 7, 2004.

(51) Int. Cl. A61K 31/70; A61K 31/366; A61K 31/225; A61K 31/192

(52) U.S. Cl. 514/23; 514/460; 514/547; 514/570

(57) ABSTRACT

The invention provides a combination of sodium gamma-hydroxybutyrate (GHB) or a prodrug or an analog thereof, with a compound that inhibits the metabolism of the GHB or GHB analog in vivo, thus prolonging or enhancing the bioactivity thereof.

### RELATED APPLICATION

This application claims priority under 35 U.S.C. 119(e) from U.S. Provisional Application Ser. No. 60/607,651 filed Sep. 7, 2004, which application is herein incorporated by reference.

### BACKGROUND OF THE INVENTION

Sodium oxybate (gamma-hydroxybutyrate, GHB, FIG. 1) is a naturally occurring soporific agent that has recently been approved for the treatment of cataplexy by the Food and Drug Administration in the United States [1]. Cataplexy, one of the cardinal symptoms of narcolepsy, refers to the sudden loss of muscle tone with emotion. Cataplexy is caused by the aberrant daytime activation of the motor atonic component of rapid-eye-movement (REM) sleep that has become dissociated from its tight coupling to REM sleep [2]. Given at night, GHB appears to promote the reintegration of sleep and to prevent its dissociation and drift into the day. In this way, it is thought to reduce daytime drowsiness and cataplexy [3]. The mechanism of action of GHB at the cellular level is not well understood, but recent studies indicate that it binds tightly to a presynaptic metabotropic G-protein coupled GHB receptor present in many brain regions, and that it is also a weak, but specific agonist, at pre- and post-synaptic G-protein coupled metabotropic GABA_{B} receptors present throughout the nervous system [4]. GHB's soporific actions are absent in knockout mice lacking GABA_{B} receptors [5].

In man, the plasma half-life of GHB given orally is about 45 minutes and doses of 2.25 grams to 4.5 grams induce only about 2 to 3 hours of sleep [3, 6]. For optimal clinical effectiveness, GHB must be given twice during the night. This is cumbersome and potentially dangerous and, for this reason, a longer acting form of the drug would be clinically advantageous. Previous work in rats using tracer doses of GHB has shown that the intravenous infusion of metabolic end products of GHB oxidation such as L-gulonate can extend the plasma half-life of GHB. However, the effect of L-gulonate on the therapeutic effects of GHB, such as induced sleep time has never been investigated [7].

### SUMMARY OF THE INVENTION

The present invention provides a therapeutic method comprising administering to a mammal, such as a human, an amount of a compound of formula (I) 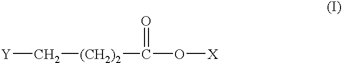
wherein X is H, a pharmaceutically-acceptable cation or (C₁-C₄)alkyl, and Y is OH, (C₁-C₄)alkoxy, (C₁-C₄)alkanoyloxy, phenylacetoxy or benzyloxy, or X and Y together form a single bond, in conjunction with an amount of an inhibitor compound that interferes with the in vivo oxidation of the compound of formula (I) so as to prolong the therapeutic effect of the compound of formula (I).

Preferably, Y is OH or (C₁-C₄)alkanoyloxy and/or X is Na⁺. A preferred compound of formula (I) is sodium gamma-hydroxybutyrate (GHB), which is available from Orphan Medical, Inc. as Xyrem®. See Physicians Desk Ref., 2416 (37ₜₕ ed. 2003). Another preferred compound of formula (I) is gamma-butyrolactone. Prodrugs of GHB, such as butane-1,4-diol are also with the scope of the invention.

One embodiment provides a therapeutic method comprising administering to a mammal an amount of a compound of formula (II) 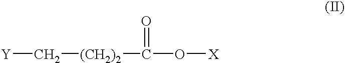
wherein X is H, a pharmaceutically acceptable cation or CO₂X represents an ester linkage to an OH group on an inhibitor compound, and Y is OH, (C₁-C₄)alkanoyloxy, phenylacetoxy or an ester linkage to a carboxylic acid group of an inhibitor compound, wherein the inhibitor compound interferes with the in vivo oxidation of the compound of formula (II) so as to prolong the therapeutic effect of the compound of formula (II).

Another embodiment provides a therapeutic method comprising administering to a mammal an amount of a compound of formula (III): 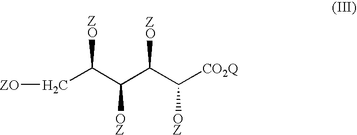
wherein each Z is H or the moiety Y—CH₂(CH₂)₂C(O)—, where at least one Z is Y—CH₂(CH₂)₂C(O)—, wherein Y is OH, (C₁-C₄)alkoxy, (C₁-C₄)alkanoyloxy, phenylacetoxy or benzyloxy, and Q is H, CH₂(CH₂)₂CO₂X or a pharmaceutically acceptable cation, wherein X is H, (C₁-C₄)alkyl or a pharmaceutically acceptable cation.

The present method can be used to treat a human afflicted with narcolepsy to reduce cataplexy and/or daytime sleepiness.

The present method can be used in humans, particularly in the elderly (>50 yrs. old), to improve the quality of sleep, or in conditions in which an increase in growth hormone levels in vivo is desired.

The present method can also be used to treat fibromyalgia or chronic fatigue syndrome, e.g., to alleviate at least one symptom of fibromyalgia or chronic fatigue syndrome.

The inhibitor compound is preferably one or more of gluconic acid lactone (GAL), glucoronic acid (GCA), glucuronic acid lactone (GCAL), gulonolactone (GL), gulonic acid (G) or a pharmaceutically-acceptable salt or esters thereof. The inhibitor compound can also include one or more of phenyl acetic acid (PA), alpha-hydroxyphenyl acetic acid, alpha-ketoglutaric acid, alpha-hydroxyglutaric acid, phenylpyruvic acid, alpha-ketoisocaproic acid, or a pharmaceutically-acceptable salt, ester or prodrug thereof. The naturally-occurring enantiomers of these compounds and their salts, prodrugs or esters are preferred for use in the present invention, as shown, for example in FIG. 1.

In some embodiments of the invention, one or more inhibitor compounds are covalently linked to sodium gamma-hydrobutyrate, via ester or ether linkages.

Therefore, the present comprises a compound of formula (II). 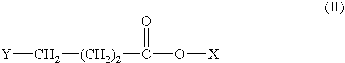
wherein X is H, a pharmaceutically acceptable cation or CO₂X represents an ester linkage to one of the OH groups on one of the inhibitor compounds, and Y is OH, (C₁-C₄)alkanoyloxy, phenylacetoxy or an ester linkage to the carboxylic acid group of one of the inhibitor compounds. Thus, the present invention includes the mono-, di- (bis), tri, tetrakis or pentakis gamma-hydroxy butyrate esters of gulonic acid, preferably, L-gulonic acid, or the pharmaceutically acceptable salts thereof. Certain of these compounds can be represented by formula (III) 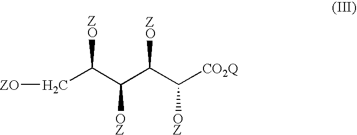
wherein each Z is individually H or Y—CH₂—(CH₂)₂—C(O)—, wherein Y is as defined above for formula (I) or (II), wherein at least one Z is Y—OCH₂(CH₂)₂—C(O)—, preferably wherein Y is H, and Q is H, (CH₂)₃CO₂X or a pharmaceutically acceptable cation, such as Na⁺, wherein X is H, a pharmaceutically acceptable cation or (C₁-C₄)alkyl.

Preferably, the inhibitor compound is present in an amount effective to reduce the ability of the compound of formula (I) or (II) to cause seizures in said mammal.

Methods of use of compounds of formulas (II) and (III) in medicine are also within the scope of the invention, e.g., as described for compound (I), as are combinations of two or more of the compounds of (I), (II) or (III).

Preferably the compound of formula (I), (II) or (III) is administered orally, separately or in admixture, preferably in combination with a pharmaceutically-acceptable carrier. The inhibitor compound is also preferably administered orally, with a carrier.

Such carriers include liquids, such as water or water/alkanol or polyol mixtures, which can optionally include buffers, flavorings and the like.

The carrier can also be a solid, to yield a tablet, pellet or capsule.

A daily dose of about 1-1000 mg/kg of the compounds of formula (I), (II) and/or (III) can be administered to accomplish the therapeutic results disclosed herein. For example, a daily dosage of about 0.5-20 g of the compound of formula (I), (II) and/or (III) can be administered, preferably about 1-15 g, in single or divided doses. For example, useful dosages and modes of administration are disclosed in U.S. Pat. Nos. 5,990,162 and 6,472,432. Methods to extrapolate from dosages found to be effective in laboratory animals such as mice, to doses effective in humans are known to the art. See U.S. Pat. No. 5,294,430.

The inhibitor compound can be administered orally or parenterally and is preferably administered before administration of the compound of formula (I). However, in some instances, the inhibitor compound is administered at the same time as the compound of formula (I), e.g., in combination or in admixture with the compound of formula (I).

The inhibitory compound can be administered in an amount effective to maintain a therapeutic level of the compound of formula (I) in the CNS or PNS of said mammal, e.g., in the brain of the mammal.

The present invention also provides a liquid or a solid composition comprising an amount of compound of formula (I) in combination with an amount of one or more inhibitor compounds that act so as they modify the pharmacokinetics of the compound of formula (I), e.g., by interfering with the in vivo oxidation of the compound of formula (I) and/or the ability of the compound of formula (I) to cause seizures at the pharmaceutically effective dose. While the inhibitor compound is preferred for use with a compound of formula (I), it can also be used to augment the action of a compound of formula (II) or (III).

### BRIEF DESCRIPTION OF THE FIGURES

FIG. 1 depicts the structures of sodium gamma-hydroxybutyrate (GHB) and the structures of certain inhibitor compounds. See www.chemfinder.com.

FIG. 2 depicts the metabolism of GHB in the cytosol and mitochondria.

FIG. 3 depicts the pentose phosphate pathway and glucuronate pathway.

FIG. 4 depicts effects of gluconic acid lactone (GAL), glucuronic acid (GCA), and gluconic acid (GA) on GHB induced sleep time (n=6/group). The mice were not fasting. The doses of all compounds were 800 mg/kg (i.p.). The data are expressed as the mean ±S.E.M. (The value was significantly higher than GHB control (P<0.001)).

FIG. 5 depicts the average hourly core body temperature during the 12 h recording period. The bar at the top of the figures indicate the room lighting condition.

FIG. 6 depicts the average hourly locomotor activity (LMA) during a 12 h recording period for the five experimental conditions. Dosing occurred during the first half of ZT19. The bar at the top of the figure indicates the room lighting condition.

FIG. 7 depicts the average hourly percentage of time spent in the spike and wave (SW) activity state during a 12 h recording period for the five experimental conditions. Dosing occurred during the first half of ZT19. The bar at the top of the figure indicates the room lighting condition.

FIG. 8 depicts the average hourly percentage of time spent awake (W). A) W during the 12 h recording period. B) W during the first 6 h of the recording period. Dosing occurred during the first half of ZT19. The bar at the top of the figures indicate the room lighting condition.

FIG. 9 depicts the average hourly wake bout duration during a 12 h recording period for the five experimental conditions. Dosing occurred during the first half of ZT19. The bar at the top of the figure indicates the room lighting condition. No significant differences were found.

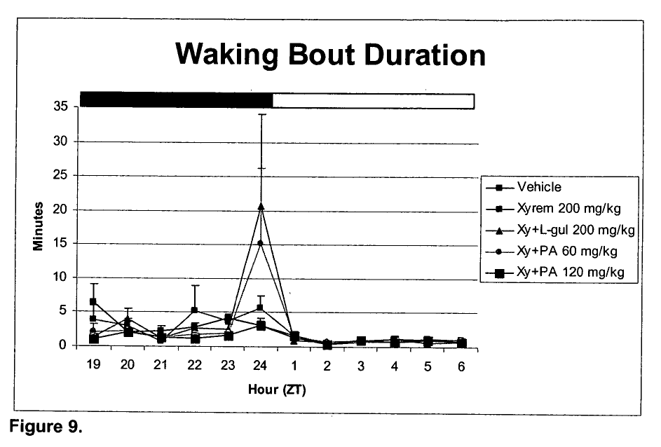

FIG. 10 depicts the average hourly percentage of time spent in non-REM (NR) sleep. A) NR during the 12 h recording. B) NR during the first 6 h of the recording. Dosing occurred during the first half of ZT19. The bar at the top of the figure indicates the room lighting condition.

FIG. 11 depicts the average hourly NR bout duration during the 12 h recording for the five experimental conditions. Dosing occurred during the first half of ZT19. The bar at the top of the figure indicates the room lighting condition.

FIG. 12 depicts the average hourly percentage of time spent in rapid eye movement (REM) sleep. A) REM during the 12 h recording. B) REM during the first 6 h of the recording. Dosing occurred during the first half of ZT19. The bar at the top of the figure indicates the room lighting condition.

FIG. 13 depicts the average hourly REM bout duration during a 12 h recording period for the five experimental conditions. Dosing occurred during the first half of ZT19. The bar at the top of the figure indicates the room lighting condition.

FIG. 14 depicts the average hourly number of REM bouts during a 12 h recording period for the five experimental conditions. Dosing occurred during the first half of ZT19. The bar at the top of the figure indicates the room lighting condition.

FIG. 15 depicts the cumulative amount of A) waking, B) NR sleep, and C) REM sleep for the first six hours of the recording period. Dosing occurred during the first half of ZT19. The bar at the top of panel A indicates the room lighting condition for all three panels.

### DETAILED DESCRIPTION OF THE INVENTION

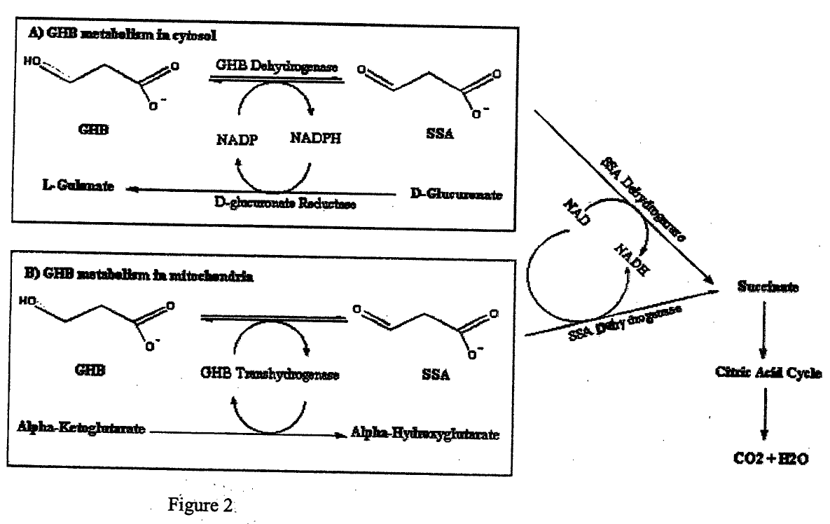

L-gulonate is generated when GHB is oxidized in the cytoplasm to succinic semialdehyde in a reaction catalyzed by GHB dehydrogenase, a member of the aldehyde reductase family of enzymes (FIG. 2) [7]. The oxidation of GHB is coupled to the reduction of glucuronic acid to gulonic acid. In mitochondria, on the other hand, a transhydrogenase couples the oxidation of GHB to the reduction of alpha-ketoglutarate to hydroxyglutarate (FIG. 2) [7]. This suggests that gulonic acid as well as hydroxyglutarate could augment the sleep promoting actions of GHB. Past studies have also shown that a number of biological intermediates resembling in some aspects the structures of known substrates of the aldehyde reductases can inhibit the oxidation of GHB. Compounds that possess inhibitory properties consist of short chain carboxylic acid intermediates of glycolysis, the Krebs cycle and fatty acid metabolism that have an alpha-keto group, a branched chain or a phenyl group. Examples of such compounds are alpha-ketoglutarate, alpha-ketoisocaproate, phenylacetate, phenylpyruvate, and hydroxyphenylpyruvate [7].

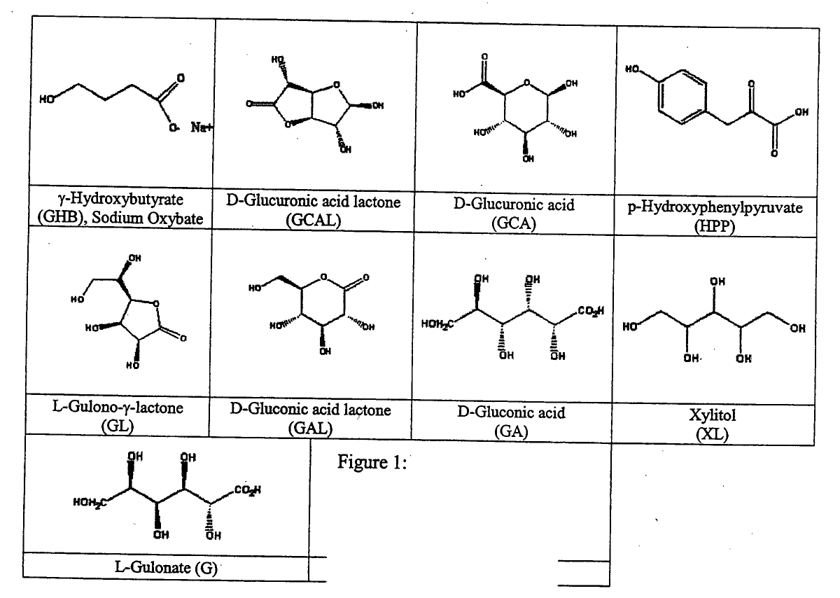

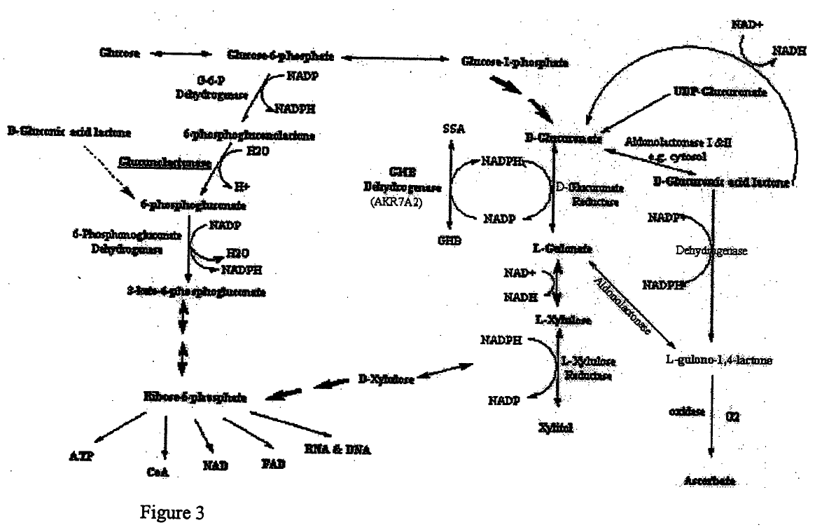

The first embodiments of the invention were developed in animal studies using D-glucuronic acid and D-gluconic acid and their lactones, and the lactone of gulonic acid (FIG. 1). All of these agents are available commercially. L-gulonic acid and D-gluconic acid are stereoisomers and both are intermediates of the pentose phosphate shunt and its auxiliary glucuronate pathway. Their structures and metabolic pathways are illustrated in FIGS. 1 and 3.

Initially the optimal dose of GHB was determined that is required to induce sleep in mice. Sleep time was measured with a passivity test and motor activity was documented with the Rota-rod. The changes in the duration of sleep and motor activity when the metabolic intermediates and their lactones were given in conjunction with GHB were then examined. Preliminary studies with phenylacetate were also conducted. Finally, a series of studies were carried out in which solutions of GHB mixed together with equimolar quantities of gulonate, gulonolactone, or gluconolactone were given orally by gavage and the effects on the duration of sleep and on motor activity in mice were compared with the effects on these parameters of GHB alone.

These preliminary studies demonstrated that L-gulonate as well as other intermediates of the pentose phosphate and auxiliary pathways and their lactones can significantly extend and alnost double the sleep time in mice produced by GHB alone. These compounds given together with GHB can also significantly augment and extend GHB's motor inhibitory actions, which suggest their utility when GHB is employed to treat cataplexy or other disorders treatable with GHB.

Pharmaceutically acceptable salts of compound (I), (II) or (III) or the inhibitor may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula (I), (II) or (III) and inhibitor compounds can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 80% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices. Unexpectedly, preservatives such as anti-microbial agents were found not to be required to render aqueous solutions of the present compounds free of microbial growth, particularly at effective concentrations of GHB greater than about 275-300 mg/ml.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are disclosed in Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula (I), (II) or (III) can be determined by comparing their in vitro activity, and in vivo activity in animal models. Methods for the extrapolation of effective dosages in mice, and other animals, to humans are known to the art; for example, see U.S. Pat. No. 4,938,949.

The invention will be further described by reference to the following detailed examples, wherein gamma-hydroxybutyrate sodium salt, D-glucuronic acid sodium salt, D-glucuronic acid lactone, D-gluconic acid sodium salt, D-gluconic acid lactone, L-gulono-γ-lactone, and phenylacetic acid were purchased from Sigma and from Aldrich. L-gulonic acid sodium salt was synthesized according to the method described by Cooper [10].

List of Abbreviations Used Herein

Rapid Eye Movement (REM); gamma-hydroxybutyrate (GHB); gluconic acid lactone (GAL); glucuronic acid (GCA); glucuronic acid lactone (GCAL); gulonolactone (GL); gluconic acid (GA); hydroxyphenylpyruvate (HPP); xylitol (XL); gulonate or gulonic acid (G); phenylacetic acid (PA); locomotor activity (LMA); zeitgeber hour (ZT); spike and wave (SW); awake or waking (W); non-REM (NR); hour (h); second (s); core body temperature (T_{b}); intraperitoneal or intraperitoneally (i.p. or IP); electroencephalograph (EEG); electromyograph (EMG); spike spindle (SS); NR delta power (NRD); W bout duration (WBD); REM bout duration (REMBD); number of REM bouts (REMNB); NR bout duration (NRBD); area postrema (AP); and mediolateral (ML).

### EXAMPLES

### Example 1

### Optimal Dose of GHB to Induce Sleep

Testing methods

(i) Animals

CD-1 male mice (30-40 g) were housed 3 to 4 per cage in the animal care facility on a 12 hour light dark cycle with free access to water and food for at least 1 week before testing.

(ii) The passivity test

The passivity test developed by Irwing was used to determine sleep time [11]. After GHB administration, the mice were placed in an unusual position and a score of 2, 4, 6, or 8 was given when the mice ceased to struggle against respectively being suspended vertically, rotated horizontally onto their backs, suspended by their hind limbs or suspended by their forelimbs. Scores on the passivity test were determined every 10 minutes after GHB administration. A score of 8 indicated that the mice were asleep. A score of 2 indicated that the mice had woken up. The time between scores of 8 and 2 was defined as the total sleep time.

(iii) Rota-Rod Measure of Motor Activity

The Rota-rod is a validated and sensitive tool that has been developed to document neurological deficits after pharmacological treatments [12, 13, 14]. In this test, mice are placed on an accelerating Rota-rod whose revolutions per minute increase from 4 to 40 cycles per minute in about 4 minutes. The time it takes for the mice to fall off the rod is the end point of the test. Initially, the mice were given 3 practice runs on the rod to familiarize themselves with the test procedure. The time that mice remained on the rod was usually between five and ten minutes. A baseline measure was then made, the test drug given and the test procedure run every 20 minutes starting at time 0, immediately after drug administration. The mice were tested for 160 minutes after GHB administration although in some experiments recordings were made for as long as 280 minutes. Motor activity was expressed as a percentage of the baseline value. The time it took the mice to fall off the rod at a given time point after drug administration was divided by the time it took at baseline.

(iv) Statistical Analysis

Statistical significance between control and treated groups at each time point in the present study was determined by student's t-test. Note that values of motor activity were expressed as a percentage of the baseline value which was normalized to 100%.

(v) Optimal Dose of GHB to Induce Sleep

The optimal dose of GHB for inducing sleep was determined with the passivity test. GHB was dissolved in 0.9% NaCl and 1% Tween 80 and delivered intraperitoneally (i.p.) to 4 groups of 6 mice that had not been fasting. Each group received either 200 mg/kg, 400 mg/kg, 600 mg/kg and 800 mg/kg. A dose of 800 mg/kg (6.4 mM/kg) reliably induced sleep within 10 minutes (Table 1). For this reason, this dose was used in the initial exploratory studies.

TABLE 1
Passivity test scores

| GHB Dose (mg/kg) | Scores (2-8) |
|---|---|
| 0 | 0 |
| 200 | 0 |
| 400 | 2 ± 0.38 |
| 600 | 4 ± 0.25 |
| 800 | 8 ± 0.42 |

Passivity test scores were recorded 10 minutes after injecting different concentration of GHB (i.p.). There were 6 non-fasting mice at each dose. Note: 0-normal, 8-no struggle.

### Example 2

### Inhibition Compounds Prolong GHB-Induced Sleep Time in Passivity Test

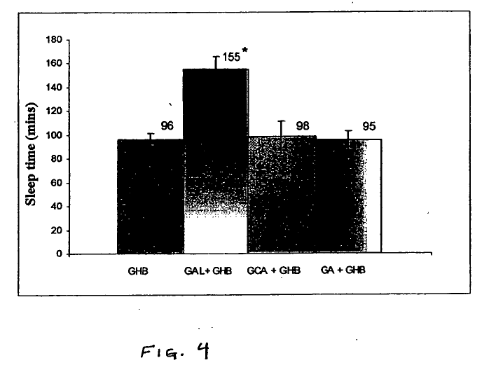

In the first study, the passivity test was used to determine whether gluconic acid lactone, glucuronic acid, or gluconic acid could prolong the sleep time produced by GHB (FIG. 4). Twenty-four mice that had not been fasting were divided into 4 groups. A control group of 6 mice received 800 mg/kg GHB i.p. and the other 3 groups of 6 mice each received 800 mg/kg of either gluconic acid lactone, glucuronic acid, or gluconic acid i.p., followed immediately by a second i.p. injection of GHB, 800 mg/kg. Mice injected with either glucuronic acid or gluconic acid did not show an increase in GHB induced sleep time. However, mice injected with gluconic acid lactone slept 155±11 minutes compared to 96.5±5 minutes with GHB alone (P<0.001).

### Example 3

### Inhibitor Compounds-Effect on Motor Activity Affected by GHB

In this experiment, gluconic acid lactone and glucuronic acid, both at 800 mg/kg, were injected 2 minutes and 15 minutes prior to the i.p. injection of GHB, 800 mg/kg, and the effect on motor activity was determined in mice that had not been fasting (Table 2).

TABLE 2
The motor performance of mice on the Rota-rod

| | % of motor | | | | | | | | | |
|---|---|---|---|---|---|---|---|---|---|---|
| | activity | Percent of motor activity after GHB administration | | | | | | | | |
| Compounds | before injection | 0′ | 20′ | 40′ | 60′ | 80′ | 100′ | 120′ | 140′ | 160′ |
| Control (vehicle) | 100 ± 12 | 93 ± 5 | 92 ± 3 | 103 ± 14 | 86 ± 4 | 86 ± 3 | 86 ± 5 | 103 ± 15 | 105 ± 15 | 100 ± 15 |
| GHB | 100 ± 20 | 31 ± 7 | 0 ± 0 | 0 ± 0 | 0 ± 0 | 20 ± 2 | 35 ± 12 | 65 ± 5 | 113 ± 20 | 124 ± 20 |
| GAL 2′ + GHB | 100 ± 15 | 5 ± 2 | 0 ± 0 | 0 ± 0 | 0 ± 5 | 0 ± 4 | 0 ± 2 | 3 ± 0*** | 4 ± 1** | 5 ± 3** |
| GAL 15′ + GHB | 100 ± 10 | 4 ± 0 | 0 ± 0 | 0 ± 0 | 0 ± 0 | 0 ± 0 | 0 ± 0* | 4 ± 0*** | 4 ± 2** | 15 ± 1** |
| GCA 2′ + GHB | 100 ± 10 | 42 ± 4 | 0 ± 1 | 0 ± 0 | 1 ± 0 | 0 ± 7 | 10 ± 1 | 11 ± 1*** | 39 ± 4* | 88 ± 4 |
| GCA 15′ + GHB | 100 ± 12 | 61 ± 1 | 1 ± 0 | 1 ± 0 | 0 ± 0 | 2 ± 0 | 7 ± 0 | 21 ± 2*** | 100 ± 10 | 141 ± 14 |
| GCAL 2′ + GHB | 100 ± 20 | 21 ± 3 | 0 ± 2 | 0 ± 0 | 0 ± 0 | 4 ± 2 | 26 ± 3 | 57 ± 10 | 58 ± 5 | 75 ± 6 |

The motor performance of mice (n = 6/group) treated i.p. with gluconic acid lactone (GAL), glucuronic acid (GCA), glucuronic acid lactone (GCAL), and GHB.
2′ or 15′ represented the preincubation time of GAL, GCA, & GCAL. The data are expressed as the mean ± S.E.M. The mice were not fasting. The doses of all compounds were 800 mg/kg.
The values with symbols *, **, and *** were significantly different than with GHB alone (P < 0.05, 0.01, 0.001, respectively).

One trial was conducted with glucuronic acid lactone given 2 minutes before GHB. Again a dose of 800 mg/kg i.p. was administered. The study was designed to determine whether there was any potential advantage to preincuabtion of the putative inhibitors of GHB metabolism. Gluconic acid lactone most effectively potentiated GHB induced motor inhibition with significant effects at the 160 minute mark whether given 2 minutes or 15 minutes before GHB. But, surprisingly, glucuronic acid, the precursor of gulonic acid, but not its lactone, also potentiated and prolonged the motor inhibition produced by GHB whether given 2 minutes or 15 minutes before GHB. However, the effect of glucuronic acid was not as long lasting as gluconic acid lactone and was not evident at 160 minutes.

### Example 4

### Inhibitor Compounds-Effect on GHB-Induced Motor Inhibition

It was next sought to determine whether the duration of motor inhibition produced by GHB could be prolonged or intensified by agents which inhibited GHB dehydrogenase rather than by intermediates of the pentose phosphate or auxiliary shunts. A single preliminary study was conducted in which the effect of phenylacetic acid, 66 mg/kg, (0.48 mmol/kg) mixed together in a solution with GHB, 800 mg/kg, (6.34 mmol/kg) and delivered orally was compared against a solution of GHB, 800 mg/kg, given orally alone (Table 3). The result showed that phenylacetic acid significantly intensified and prolonged the inhibitory effects of GHB on motor activity.

TABLE 3
The motor performance of mice on the Rota-rod

| | % of Motor | | | | | | | | | |
|---|---|---|---|---|---|---|---|---|---|---|
| | activity | Percent of motor activity after GHB administration (oral) | | | | | | | | |
| Compounds (oral) | before injection | 0′ | 20′ | 40′ | 60′ | 80′ | 100′ | 160′ | 220′ | 280′ |
| GHB | 100 ± 0 | 51 ± 1 | 0 ± 0 | 7 ± 3 | 12 ± 6 | 20 ± 8 | 26 ± 5 | 86 ± 1 | 90 ± 8 | 100 ± 7 |
| PA + GHB | 100 ± 0 | 60 ± 4 | 1 ± 1 | 1 ± 1 | 1 ± 1 | 3 ± 1 | 5 ± 0** | 45 ± 12** | 75 ± 14 | 85 ± 9 |

Preliminary study of the motor performance of mice (n = 6/group) given phenylacetic acid (PA) orally together with GHB compared to GHB orally alone.
The data are expressed as the mean ± S.E.M. The mice were fasting for 12 hours. The dose of GHB was 800 mg/kg and the dose of PA was 66 mg/kg (0.48 mmol/kg).
The values with symbol ** were significantly different than with GHB alone (P < 0.01).

### Example 5

### Parenterally-Administered Inhibitory Compounds

Because of concerns that the oral bioavailability of 800 mg/kg GHB in mice that had not been fasting was insufficient to reliably induce sleep, an exploratory study was conducted with GHB orally at a dose of 1200 mg/kg alone and in combination with either gluconic acid lactone, gulonolactone, or gluconic acid, all at 1200 mg/kg i.p. (Table 4).

TABLE 4
The motor performance of mice on the Rota-rod

| | % of Motor | | | | | | | | |
|---|---|---|---|---|---|---|---|---|---|
| | activity | Percent of motor activity after GHB administration | | | | | | | |
| Compounds | before injection | 0′ | 20′ | 40′ | 60′ | 80′ | 100′ | 160′ | 220′ |
| GHB | 100 ± 0 | 58 ± 9 | 27 ± 19 | 0 ± 0 | 3 ± 2 | 4 ± 1 | 5 ± 4 | 21 ± 10 | 61 ± 12 |
| GAL + GHB | 100 ± 15 | 24 ± 4 | 0 ± 0 | 0 ± 0 | 0 ± 0 | 0 ± 0 | 0.67 ± 0 | 0 ± 7 | 0 ± 0*** |
| GL + GHB | 100 ± 23 | 50 ± 5 | 0 ± 0 | 0 ± 0 | 0 ± 0 | 0 ± 0 | 2.5 ± 8 | 0 ± 0 | 7 ± 4** |
| GA + GHB | 100 ± 0 | 98 ± 13 | 14 ± 10 | 0 ± 0 | 7 ± 2 | 50 ± 7 | 80 ± 9 | 100 ± 30 | 76 ± 10 |

The motor performance of the mice (n = 6/group) after the coadministration of GHB orally and gluconic acid lactone (GAL), gulonolactone (GL), gluconic acid (GA) intraperitoneally.
The data are expressed as the mean ± S.E.M. The mice were fasting for 1 hour. The doses of all the compounds were 1200 mg/kg.
The values with symbols **, and *** were significantly different than with GHB alone (P < 0.01 & 0.001, respectively).

Oral GHB at 1200 mg/kg had a more prolonged effect and depressed motor activity for at least 220 minutes but this effect was further significantly enhanced by both gluconic acid lactone and gulonolactone (P<0.001 and P<0.01, respectively). Again, as on the initial passivity test, gluconic acid lactone was the most effective.

### Example 6

### Effect of L-Gulonate on GHB Activity

In this study, GHB was given orally to 8 mice in a dose of 1000 mg/kg or 7.93 mmol/kg. A dose of 1000 mg/kg GHB was used rather than 1200 mg/kg because 1200 mg/kg appeared to induce seizure-like activity in many of the mice. As shown in Table 6, 1000 mg/kg GHB produced a reproducible decrease in motor activity lasting more than 100 minutes. Thus, a dose of 1000 mg/kg appeared to be optimal.

For this reason, GHB 7.93 mmol/kg (1000 mg/kg) was mixed in solution with either 7.93 mmol/kg gluconic acid lactone, L-gulonolactone, or L-gulonate sodium salt and was given orally by gavage to each of 3 groups of 8 mice. The effects on sleep time and on motor inhibition were then determined. All three agents appeared to increase the sleep time but the effect only reached significance with the sodium salt of L-gulonate. As shown on Table 5, this agent almost doubled the sleep time.

TABLE 5

| Compounds | Sleep time (mins) |
|---|---|
| GHB | 35 ± 10 |
| GHB + GAL | 63 ± 26 |
| GHB + GL | 58 ± 6 |
| GHB + G | 68 ± 10* |

The sleep time of the mice (n = 8/group) after GHB and gluconic acid lactone (GAL), gulonolactone (GL), or L-gulonate (G) administration (orally).
The data are expressed as the mean ± S.E.M. The mice were fasting for 24 hours. The doses of all compounds were 7.93 mmol/kg.
The value with symbols * was significantly different than with GHB alone (P < 0.05).

L-gulonate sodium salt on its own had no effect on motor performance but this agent significantly prolonged and deepened the motor inhibition produced by GHB (Table 6). In this study on motor activity, neither oral gluconic acid lactone or gulonolactone augmented the depressant effects of GHB.

TABLE 6
The motor performance of the mice on Rota-rod

| | % of motor | | | | | | | | | |
|---|---|---|---|---|---|---|---|---|---|---|
| | activity | Percent of motor activity after GHB administration | | | | | | | | |
| Compounds | before injection | 0′ | 20′ | 40′ | 60′ | 80′ | 100′ | 160′ | 220′ | 280′ |
| GHB | 100 ± 0 | 94 ± 9 | 2 ± 2 | 0 ± 0 | 4 ± 1 | 10 ± 2 | 22 ± 7 | 104 ± 26 | 122 ± 23 | 117 ± 13 |
| GHB + GAL | 100 ± 0 | 70 ± 9 | 10 ± 5 | 4 ± 3 | 6 ± 3 | 8 ± 5 | 7 ± 3 | 97 ± 10 | 105 ± 5 | 104 ± 4 |
| GHB + GL | 100 ± 0 | 52 ± 11 | 2 ± 1 | 4 ± 3 | 4 ± 2 | 4 ± 2 | 12 ± 4 | 78 ± 9 | 93 ± 10 | 102 ± 7 |
| G | 100 ± 0 | 168 ± 5 | 50 ± 24 | 303 ± 12 | 183 ± 4 | 156 ± 2 | 66 ± 9 | 126 ± 3 | 303 ± 23 | 141 ± 5 |
| GHB + G | 100 ± 0 | 74 ± 12 | 1 ± 0 | 1 ± 1 | 0 ± 0*** | 3 ± 1* | 3 ± 1 | 13 ± 3* | 45 ± 11** | 98 ± 14 |

The motor performance of mice (n = 8/group) coadministered (orally) with gluconic acid lactone (GAL), gulonolactone (GL), or L-gulonate (G) with GHB.
The data are expressed as the mean ± S.E.M. The mice were fasting for 24 hours. The doses of all compounds were 7.93 mmol/kg.
The values with symbols *, **, and *** were significantly different than with GHB alone (P < 0.05, 0.01, & 0.001, respectively).

### Results for Examples 1-6

The aforementioned studies reveal that inhibitors of GHB dehydrogenase and metabolic intermediates of the pentose phosphate and auxiliary glucuronate pathways can prolong and augment the sleep inducing and motor inhibitory effects of GHB. The compounds that most effectively extend the actions of GHB are L-gulonate, D-gluconic acid lactone and gulonolactone.

D-glucuronic acid can augment the motor inhibitory effects of GHB even though Kaufman and Nelson [7] reported that D-glucuronate decreased the plasma half-life of GHB. In the present examples, D-glucuronic acid was given i.p. 2 minutes and 15 minutes prior to GHB administration. This route might allow sufficient time for D-glucuronic acid to be transformed to L-gulonic acid.

D-Gluconic acid lactone also effectively prolonged the duration of action of GHB. The lactone of gluconic acid is readily converted to 6-phosphogluconate and is then oxidized by NADP and 6-phosphogluconate dehydrogenase in the pentose phosphate pathway to form 3-keto-6-phosphogluconate (FIG. 3) [8, 9]. D-gluconic acid lactone and L-gulonate may both compete with GHB for the co-factor NADP and, in this way, D-gluconic acid lactone may also inhibit GHB dehydrogenase activity and prolong the action of GHB.

Phenylacetic acid, a direct inhibitor of GHB dehydrogenase, can also prolong the motor inhibitory actions of GHB. A dose of 66 mg/kg (0.48 mmol/kg) of the phenylacetic acid was used because this was the greatest quantity that would dissolve in a saline solution. However, phenylacetate sodium salt, can be readily dissolved in saline and should be useful in future studies [15]. In any case, phenylpyruvate and hydroxyphenylpyruvate can be even more effective than phenylacetate because these compounds contain both α-keto group and a phenyl group. The naturally occurring inhibitors of GHB dehydrogenase may also have a practical advantage over pentose phosphate shunt intermediates in terms of drug design because they can effectively augment the actions of GHB at very low concentrations.

### Example 7

### Effect of GHB+L-Gulonate and GHB+phenyl acetate on Sleep in Rats

In this study, the formulations of GHB+L-gulonate (L-gul) and GHB+phenyl acetate (PA) were tested in rats for their effects on sleep parameters, core body temperature (T_{b}) and locomotor activity (LMA). The experimental formulations were compared to GHB alone and a vehicle control. Using a randomized, repeated measures design, the effects of GHB+L-gul and GHB+PA on both sleep/wake amounts as well as sleep consolidation parameters (bout duration and number of bouts per h) were investigated.

### Materials and Methods

Animal Recording and Surgical Procedures

Animals were housed in a temperature controlled recording room under a 12/12 light/dark cycle (lights on at 7:00 am) and had food and water available ad libitum. Room temperature (24±2° C.), humidity (50±20% relative humidity), and lighting conditions were monitored continuously via computer.

Eight male Wistar rats (300±25 g; Charles River, Wilmington, Mass.) were prepared with chronic recording implants for continuous electroencephalograph (EEG) and electromyograph (EMG) recordings. Under isofluorane anesthesia (1-4%), stainless steel screws (#000) were implanted into the skull and served as epidural electrodes. EEG electrodes were positioned bilaterally at +2.0 mm AP from bregma and 2.0 mm ML, and at −6.0 mm AP and 3.0 mm ML. Multi-stranded twisted stainless steel wire electrodes were sutured bilaterally in the neck muscles for recording of the EMG. EMG and EEG electrodes were soldered to a head plug connector that was affixed to the skull. The incisions were sutured, and antibiotics were administered topically. Following completion of the skull implantation, miniature transmitters (E-mitters, MiniMitter, Bend, Oreg., U.S.A.) were implanted for continuous T_{b} and LMA recordings. The cold sterilized (Cidex) transmitter was inserted into the peritoneum and sewn to the musculature. Furacin ointment was applied to the sutured incision. Pain was relieved by a long-lasting analgesic (Buprenorphine) administered intramuscularly post-operatively. Post-surgery, animals were placed in clean cages and observed until they recovered. Animals were permitted a minimum of one week post-operative recovery before study.

For sleep recordings, animals were connected via a cable and a counter-balanced commutator to a Neurodata model 15 data collection system (Grass-Telefactor, West Warwick, R.I., U.S.A.). The animals were allowed an acclimation period of at least 48 h before the start of the experiment and were connected to the recording apparatus continuously throughout the experimental period, except to replace damaged cables. The amplified EEG and EMG signals were digitized and stored on a computer using SleepSign software (Kissei Comtec, Irvine, Calif., U.S.A.). T_{b} and LMA were recorded and stored on a computer using VitalView software (MiniMitter, Bend, Oreg., U.S.A.).

Experimental Design

Three novel formulations of GHB were tested for their effects on sleep parameters, and were compared to GHB alone and vehicle control.

TABLE 7
Experimental Doses

| | |
|---|---|
| GHB + L-gul | 200 mg/kg GHB + 200 mg/kg L-gulonate |
| GHB + PA 60 mg/kg | 200 mg/kg GHB + 60 mg/kg Phenylacetate |
| GHB + PA 120 mg/kg | 200 mg/kg GHB + 120 mg/kg Phenylacetate |
| GHB | 200 mg/kg GHB |
| vehicle control | Saline |

A repeated measures design was employed in which each rat was to receive six separate intraperitoneal (IP) dosings. The first dosing was comprised only of vehicle and was used to acclimate the rats to the dosing procedures. The second through sixth dosings were the five dosing conditions described above and given in randomized order. Since all dosings were administered while the rats were connected to the recording apparatus, 60% CO₂/40% O₂ gas was employed for light sedation during the dosing procedure. Rats appeared fully recovered within 60 s following the procedure. A minimum of three days elapsed between dosings. Since the test compound was hypothesized to promote sleep, dosing occurred during the middle of the rats' normal active period. The dosing procedure began approximately 6 hr after lights off during the start of zeitgeber hour 19 (ZT19) and was typically completed by the middle of the hour. Following each dosing, animals were continuously recorded for 30 h until lights out the following day (ZT12). However, only the first 12 h of the recording were scored and analyzed.

Data Analysis

EEG and EMG data were scored visually in 10 s epochs for waking (W), rapid eye movement (REM) sleep, and nonREM (NR) sleep. Scored data were analyzed and expressed as time spent in each state per hour. In order to investigate possible effects on sleep consolidation, sleep bout duration and number of bouts for each state were calculated in hourly bins. A “bout” consisted of a minimum of two consecutive 10 s epochs of a given state and ended with any single state change epoch. EEG delta power (0.5-3.5 Hz) within NR sleep (NRD) was also analyzed in hourly bins. The EEG spectra during NR were obtained offline with a fast Fourier transform algorithm on all epochs without artifact. For each individual animal, delta power was normalized to the average delta power in NR during the last two h of the analyzed period (ZT5-6). T_{b} (° C.) and LMA (counts per min) were averaged and analyzed in hourly bins.

Data were analyzed using two-way repeated measures ANOVA. Light phase and dark phase data were analyzed separately. It was anticipated that both a treatment effect and an effect that changed over time (i.e., decreased) would occur, so both the treatment effect (factor A) and time (factor B) within each rat and the time x treatment effect within each rat were analyzed. It was necessary for at least two of these three statistics to reach statistical significance in order to designate an ANOVA result to be significant overall. When statistical significance was found from the ANOVAs, Fisher's LSD t-tests were performed.

### Results for Example 7

Body Temperature and Locomotor Activity

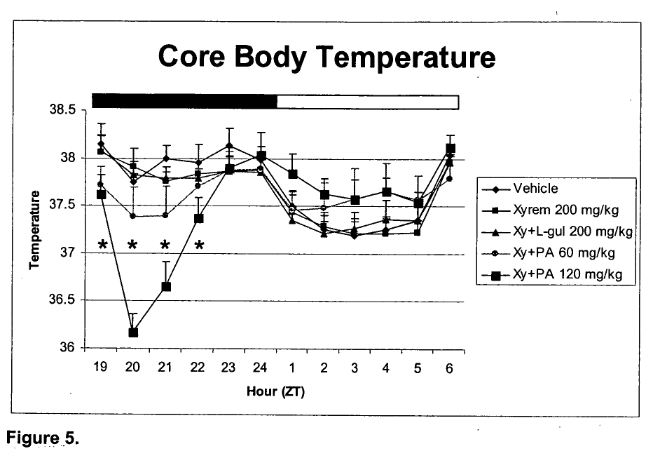

Core body temperature (T_{b}) was significantly lower following GHB+PA (60 and 120 mg/kg) compared to vehicle, GHB and GHB+L-gul (FIG. 5). Following GHB+PA (120 mg/kg), T_{b} was significantly lower than vehicle (ZT19-22), GHB (ZT19-22), GHB+L-gul (ZT19-22) and GHB+PA (60 mg/kg) (ZT20-21). T_{b} following GHB+PA (60 mg/kg) was significantly lower than vehicle (ZT19-21), GHB (ZT20-22), and GHB+L-gul (ZT20-21).

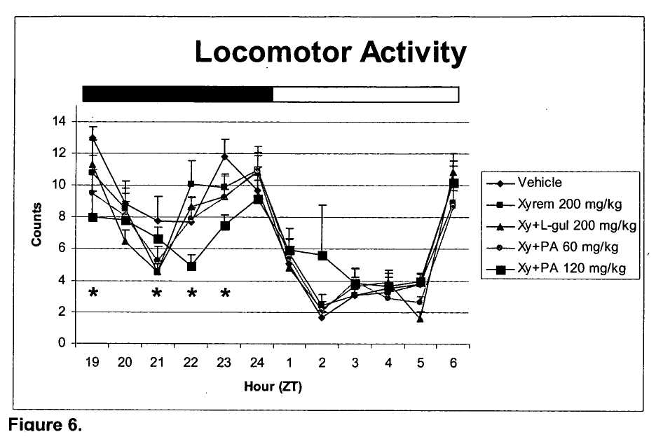

Locomotor activity (LMA) was also significantly lower following GHB+PA (60 and 120 mg/kg) (FIG. 6). Following GHB+PA (120 mg/kg), LMA was significantly lower than vehicle (ZT19, 22-23), GHB (ZT19, 22), GHB+L-gul (ZT19, 22) and GHB+PA (60 mg/kg) (ZT22). LMA following GHB+PA (60 mg/kg) was significantly lower than vehicle (ZT19, 23) only. GHB and GHB+L-gul also elicited significantly lower LMA compared to vehicle (ZT21).

Abnormal EEG Activity

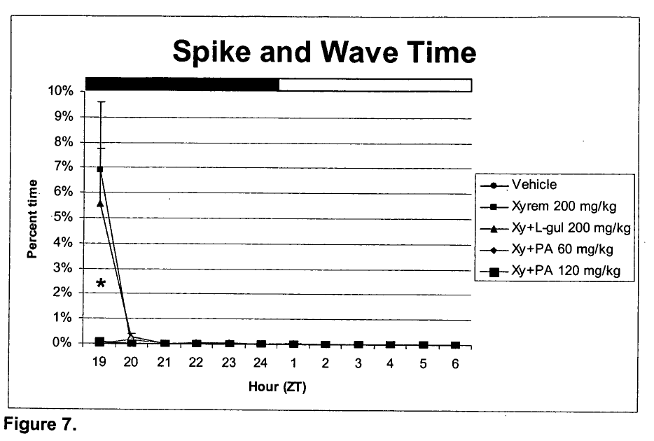

Both GHB and GHB+L-gul elicited spike and wave activity (SW) in the EEG during the first hour following dosing (FIG. 7). SW activity was not found following vehicle, GHB+PA (60 or 120 mg/kg) dosings. In addition to this SW activity, a second abnormal EEG waveform was found in some rats. Where the SW activity tended to be unipolar spiking in the 5-7 hz range, this second abnormal activity was more bipolar in form and fell in the 7-9 hz range. This activity is referred to as spindle spike (SS) activity. SS activity appeared in a very different pattern than SW activity (see Table 8). Only 4 of the 8 rats displayed SS activity. Two rats (OMT 402 and 405) displayed SS activity throughout all five experimental conditions. OMT 403 did not display SS activity during the first two dosing conditions but did throughout the final three conditions. OMT 406 only displayed SS activity during the fifth dosing condition. There was no relationship to drug condition, and once a rat displayed SS activity, it was found throughout the recording period for every subsequent condition.

TABLE 8
Percent Time in spindle-spike condition for each individual rat.
The letters below the rat ID# represent the drug dosing
condition. The row of numbers below the letters representing the
dosing condition represent the order of the dosing condition.

| | OMT402 | | | | | OMT403 | | | | | OMT404 | | | | | OMT405 | | | | |
|---|---|---|---|---|---|---|---|---|---|---|---|---|---|---|---|---|---|---|---|---|
| | B | E | A | D | C | C | E | B | A | D | A | B | D | E | C | E | A | D | C | B |
| Hour | 1 | 2 | 3 | 4 | 5 | 1 | 2 | 3 | 4 | 5 | 1 | 2 | 3 | 4 | 5 | 1 | 2 | 3 | 4 | 5 |
| 19 | 11% | 5% | 2% | 19% | 11% | 0% | 0% | 2% | 1% | 1% | 0% | 0% | 0% | 0% | 0% | 0% | 3% | 2% | 4% | 3% |
| 20 | 2% | 1% | 5% | 2% | 3% | 0% | 0% | 5% | 1% | 4% | 0% | 0% | 0% | 0% | 0% | 0% | 1% | 1% | 1% | 2% |
| 21 | 0% | 1% | 3% | 1% | 0% | 0% | 0% | 3% | 0% | 0% | 0% | 0% | 0% | 0% | 0% | 0% | 1% | 0% | 0% | 3% |
| 22 | 2% | 0% | 0% | 1% | 1% | 0% | 0% | 0% | 1% | 0% | 0% | 0% | 0% | 0% | 0% | 0% | 1% | 1% | 1% | 0% |
| 23 | 4% | 0% | 3% | 5% | 10% | 0% | 0% | 3% | 0% | 0% | 0% | 0% | 0% | 0% | 0% | 1% | 1% | 1% | 2% | 2% |
| 24 | 4% | 2% | 4% | 3% | 5% | 0% | 0% | 4% | 0% | 1% | 0% | 0% | 0% | 0% | 0% | 2% | 1% | 1% | 2% | 0% |
| 1 | 1% | 0% | 0% | 2% | 3% | 0% | 0% | 0% | 0% | 0% | 0% | 0% | 0% | 0% | 0% | 0% | 0% | 1% | 0% | 0% |
| 2 | 0% | 0% | 0% | 0% | 0% | 0% | 0% | 0% | 0% | 0% | 0% | 0% | 0% | 0% | 0% | 0% | 0% | 0% | 0% | 0% |
| 3 | 0% | 0% | 0% | 0% | 0% | 0% | 0% | 0% | 0% | 1% | 0% | 0% | 0% | 0% | 0% | 0% | 0% | 0% | 2% | 0% |
| 4 | 0% | 0% | 0% | 0% | 1% | 0% | 0% | 0% | 1% | 0% | 0% | 0% | 0% | 0% | 0% | 0% | 0% | 1% | 1% | 0% |
| 5 | 0% | 1% | 2% | 2% | 0% | 0% | 0% | 2% | 3% | 0% | 0% | 0% | 0% | 0% | 0% | 0% | 1% | 0% | 0% | 1% |
| 6 | 0% | 0% | 0% | 0% | 0% | 0% | 0% | 0% | 1% | 0% | 0% | 0% | 0% | 0% | 0% | 0% | 0% | 0% | 0% | 1% |
| | OMT406 | | | | | OMT407 | | | | | OMT408 | | | | | OMT409 | | | | |
| | D | C | B | A | E | C | B | E | A | D | D | A | E | C | B | B | C | D | E | A |
| | 1 | 2 | 3 | 4 | 5 | 1 | 2 | 3 | 4 | 5 | 1 | 2 | 3 | 4 | 5 | 1 | 2 | 3 | 4 | 5 |
| 19 | 0% | 0% | 0% | 0% | 3% | 0% | 0% | 0% | 0% | 0% | 0% | 0% | 0% | 0% | 0% | 0% | 0% | 0% | 0% | 0% |
| 20 | 0% | 0% | 0% | 0% | 2% | 0% | 0% | 0% | 0% | 0% | 0% | 0% | 0% | 0% | 0% | 0% | 0% | 0% | 0% | 0% |
| 21 | 0% | 0% | 0% | 0% | 3% | 0% | 0% | 0% | 0% | 0% | 0% | 0% | 0% | 0% | 0% | 0% | 0% | 0% | 0% | 0% |
| 22 | 0% | 0% | 0% | 0% | 0% | 0% | 0% | 0% | 0% | 0% | 0% | 0% | 0% | 0% | 0% | 0% | 0% | 0% | 0% | 0% |
| 23 | 0% | 0% | 0% | 0% | 2% | 0% | 0% | 0% | 0% | 0% | 0% | 0% | 0% | 0% | 0% | 0% | 0% | 0% | 0% | 0% |
| 24 | 0% | 0% | 0% | 0% | 0% | 0% | 0% | 0% | 0% | 0% | 0% | 0% | 0% | 0% | 0% | 0% | 0% | 0% | 0% | 0% |
| 1 | 0% | 0% | 0% | 0% | 0% | 0% | 0% | 0% | 0% | 0% | 0% | 0% | 0% | 0% | 0% | 0% | 0% | 0% | 0% | 0% |
| 2 | 0% | 0% | 0% | 0% | 0% | 0% | 0% | 0% | 0% | 0% | 0% | 0% | 0% | 0% | 0% | 0% | 0% | 0% | 0% | 0% |
| 3 | 0% | 0% | 0% | 0% | 0% | 0% | 0% | 0% | 0% | 0% | 0% | 0% | 0% | 0% | 0% | 0% | 0% | 0% | 0% | 0% |
| 4 | 0% | 0% | 0% | 0% | 0% | 0% | 0% | 0% | 0% | 0% | 0% | 0% | 0% | 0% | 0% | 0% | 0% | 0% | 0% | 0% |
| 5 | 0% | 0% | 0% | 0% | 1% | 0% | 0% | 0% | 0% | 0% | 0% | 0% | 0% | 0% | 0% | 0% | 0% | 0% | 0% | 0% |
| 6 | 0% | 0% | 0% | 0% | 1% | 0% | 0% | 0% | 0% | 0% | 0% | 0% | 0% | 0% | 0% | 0% | 0% | 0% | 0% | 0% |

Wakefulness

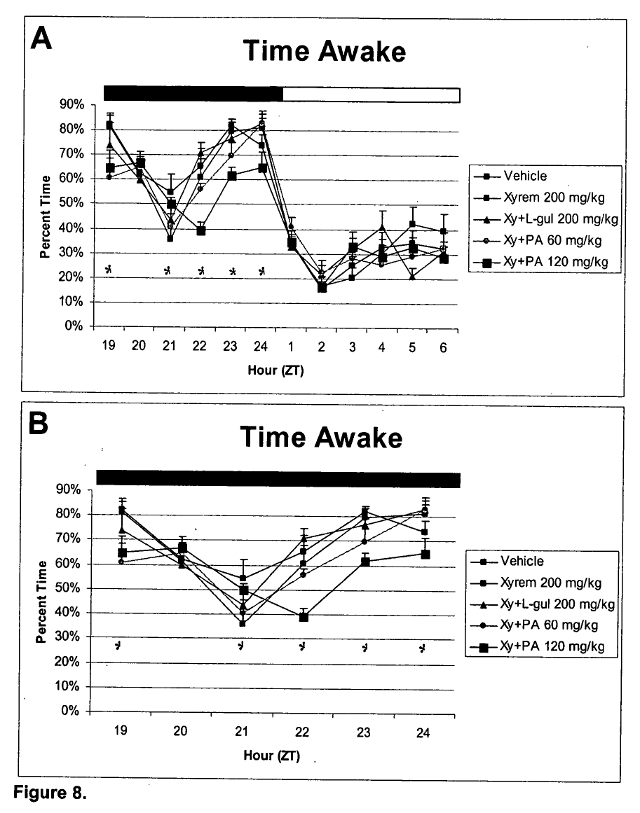

GHB+PA (120 mg/kg) produced the largest reduction in time awake (W) (FIG. 8). Following GHB+PA (120 mg/kg), W was significantly less than vehicle (ZT19, 22-23), GHB (ZT 19, 22-24), GHB+L-gul (ZT 22, 24), and GHB+PA (60 mg/kg) (ZT 22, 24). GHB reduced W compared to vehicle during ZT 21. Following GHB+PA (60 mg/kg), W was significantly less than vehicle and GHB during ZT 19.

NR Sleep and NR Delta Power

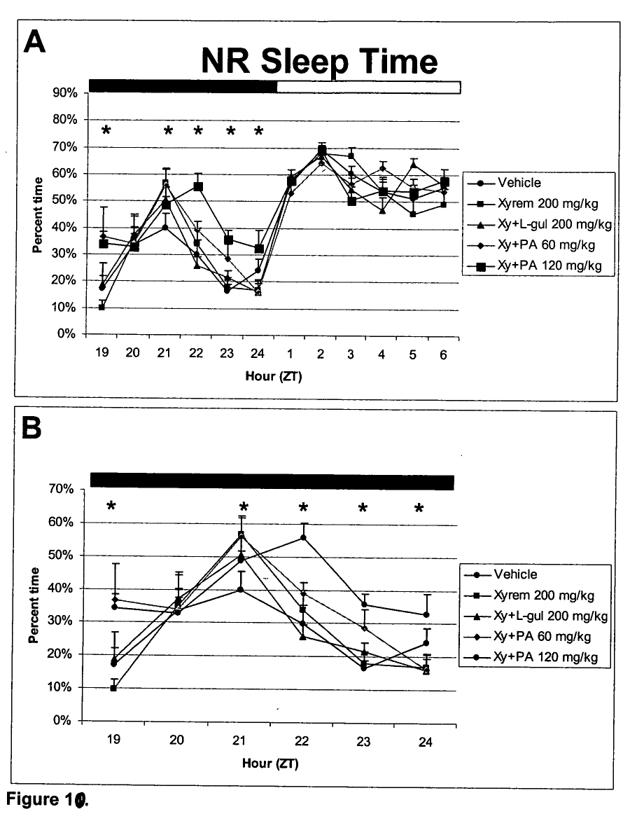

As with W, GHB+PA (120 mg/kg) produced the largest effect on non-REM (NR) sleep (FIG. 10). Following GHB+PA (120 mg/kg), NR was significantly increased compared to vehicle (ZT19, 22-23), GHB (ZT 19, 22-24), GHB+L-gul (ZT 19, 22, 24) and GHB+PA (60 mg/kg) (ZT 22, 24). GHB+PA (60 mg/kg) also significantly increased NR compared to vehicle (ZT 19, 21), GHB (ZT19) and GHB+L-gul (ZT 19). Following GHB, NR was significantly greater than vehicle during ZT 21. NRD was not significantly different across the conditions (data not shown).

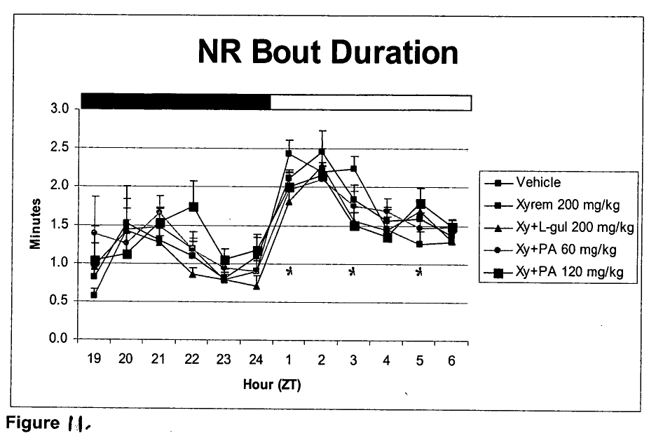

GHB produced significant changes in NR bout duration (NRBD) but only during the second half of the recording period (lights on; FIG. 11). GHB increased NRBD compared to GHB+L-gul (ZT 1, 3), GHB+PA (60 mg/kg) (ZT 1, 3) and GHB+PA (120 mg/kg) (ZT 3). GHB significantly decreased NRBD during ZT 5 compared to GHB+L-gul and GHB+PA (120 mg/kg). No significant differences were found for the number of NR bouts (data not shown).

REM Sleep

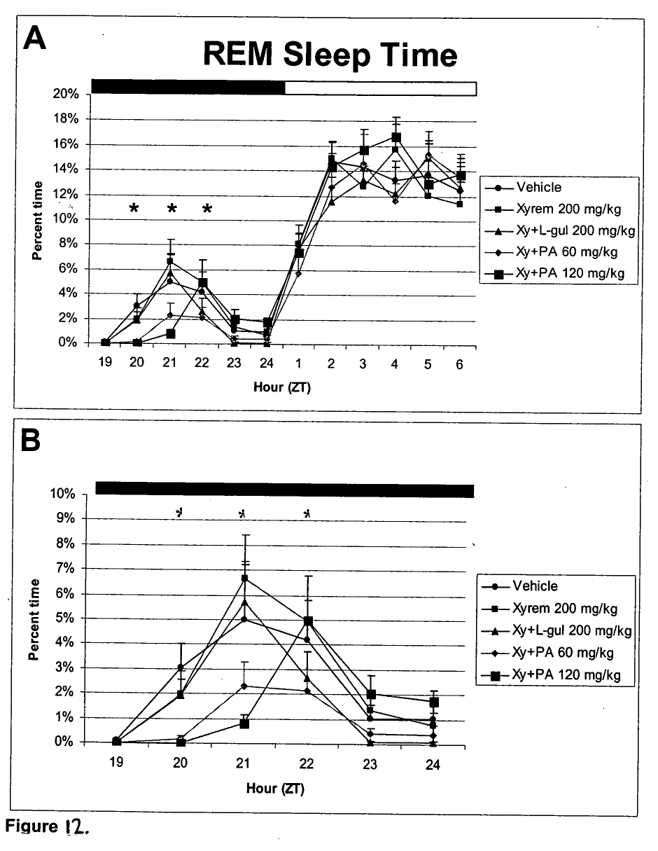

Both GHB+PA (60 and 120 mg/kg) significantly suppressed REM sleep compared to vehicle (ZT 20, 21), GHB (ZT 21) and GHB+L-gul (ZT 21) (FIG. 12). However, GHB+PA (120 mg/kg) also elicited increased REM sleep during ZT 22 compared to GHB+L-gul and GHB+PA (60 mg/kg).

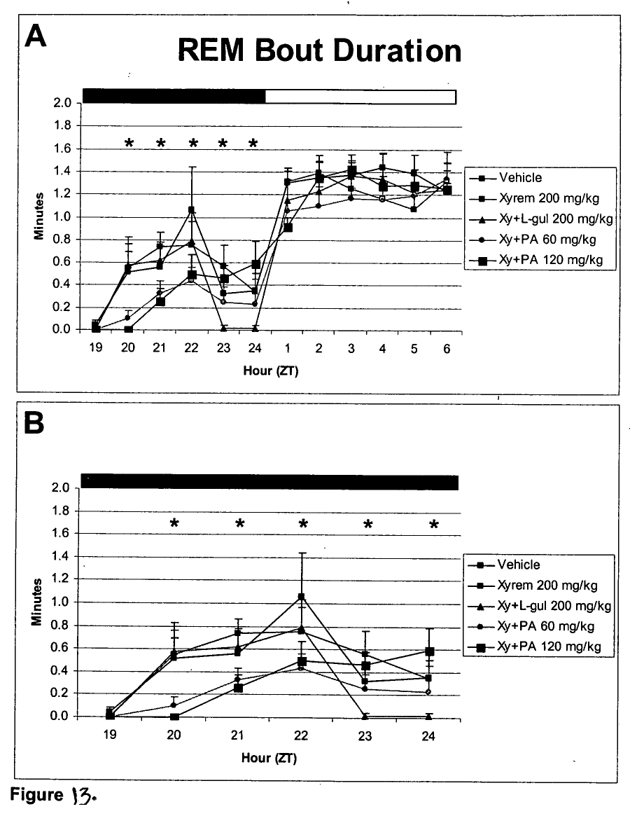

GHB+PA also decreased REM bout duration (REMBD, FIG. 13). Following GHB+PA (120 mg/kg), REMBD was significantly shorter than vehicle (ZT 20, 22), GHB (ZT20-21) and GHB+L-gul (ZT 20). Following GHB+PA (60 mg/kg), REMBD was significantly shorter than vehicle (ZT 22) and GHB+L-gul (ZT 20). GHB+L-gul elicited shorter REMBD compared to GHB (ZT 23) and GHB+PA (120 mg/kg) (ZT 24).

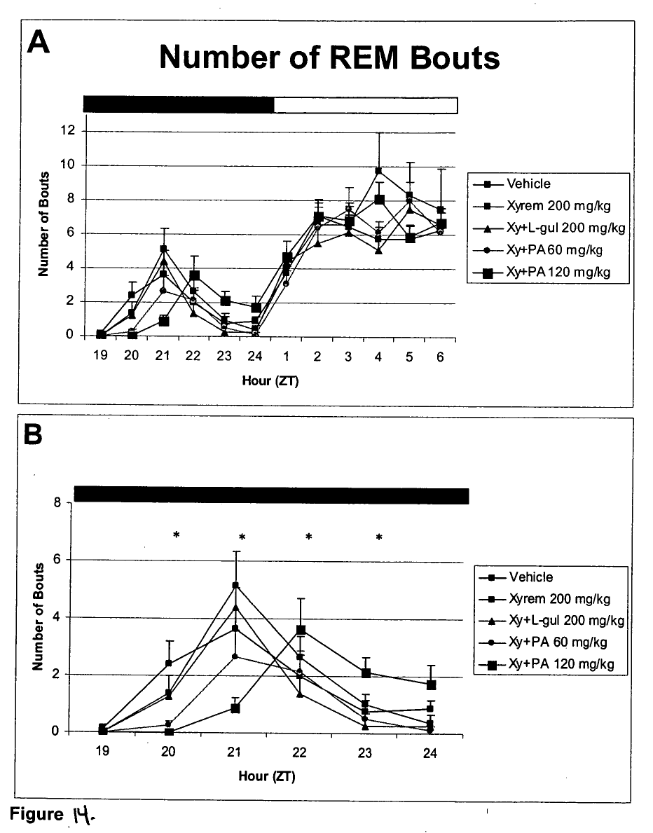

The number of REM bouts (REMNB) was also affected primarily by GHB+PA (FIG. 14). Following GHB+PA (120 mg/kg), there were fewer REM bouts compared to vehicle (ZT 20-21), GHB, (ZT 21), GHB+L-gul (ZT 21) and GHB+PA (60 mg/kg) (ZT 21). REMNB were also decreased following GHB+PA (60 mg/kg) compared to vehicle (ZT 20), GHB (ZT 21) and GHB+L-gul (ZT 21). During both ZT 23 and 24, however, there were significantly more REM bouts following GHB+PA (120 mg/kg) compared to GHB+L-gul.

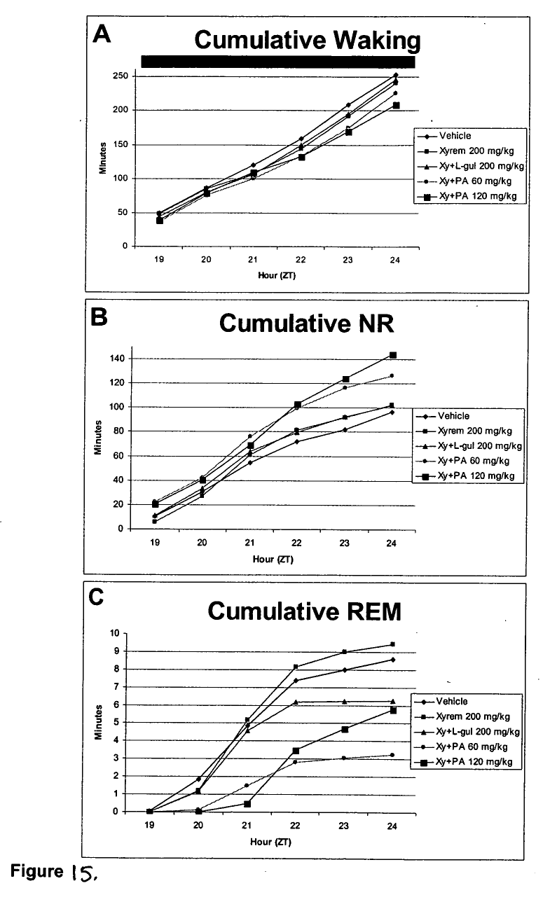

Of the four drug conditions tested, GHB+PA (120 mg/kg) was the most effective at increasing NR sleep. Following GHB+PA (120 mg/kg), W was significantly decreased and NR sleep significantly increased in 3 of the first 6 hours of the recording (ZT 19, 22 and 23) compared to vehicle. As can be seen in FIG. 15, cumulative NR sleep increased and cumulative W decreased over the first 6 hours of the recordings, as compared to vehicle. GHB+PA (60 mg/kg) had an intermediate effect on these parameters. REM sleep was suppressed by GHB+PA with the 120 mg/kg dose suppressing REM less than the 60 mg/kg dose. GHB alone significantly increased NR sleep and decreased W during ZT 21 compared to vehicle. GHB+L-gul elicited no significant differences in sleep parameters compared to vehicle.

T_{b} was also affected primarily by GHB+PA. GHB+PA (120 mg/kg) produced significant hypothermia during the first 5 hours of recording compared to vehicle. GHB+PA (60 mg/kg) also produced significant decreases in T_{b} for the first 3 hours of recording. These decreases in T_{b} were due primarily to individual rats displaying pronounced hypothermia. Following GHB+PA (120 mg/kg), 3 individual rats had T_{b} fall to 35-36° C. Following GHB+PA (60 mg/kg), the T_{b} of one rat fell to 35-36° C. The cause of this hypothermia is unknown.

Two types of abnormal EEG activity were observed. SS activity was displayed by 4 of 8 rats. Two rats displayed SS activity following all five dosing conditions and throughout the recording period. Two other rats began to display SS activity during the course of the experiment (one on dosing day 3 and one on dosing day 5). Once SS activity was displayed, it was seen during all subsequent conditions, regardless of drug condition.

SW activity was displayed immediately following the GHB and GHB+L-gul gul conditions. Seven of 8 rats displayed SW activity following GHB, and 6 of 8 rats displayed SW activity following GHB+L-gul. The SW activity displayed following both GHB and GHB+L-gul was not seen following either dose of GHB+PA, indicating that PA may play a protective role against seizure activity caused by GHB. Phenyl acetate combined with GHB also enhances the NR sleep promoting effects of GHB alone.

### Bibliography

1. Physicians Desk Reference (58^{th} ed. 2004) at 2403.

2. Guilleminault, C., Cataplexy. In: Guilleminault, C., Dement, W., Passounant, P. (eds.), Narcolepsy. New York: Spectrum, 125-143, 1976.

3. Broughton, R., Mamelak, M., Effects of nocturnal gamma-hydroxybutyrate on sleep/waking patterns in narcolepsy-cataplexy, Canadian Journal of Neurological Science, 7: 23-30, 1980.

4. Bernasconi, R. et al., Part of the pharmacological actions of gamma-hydroxybutyrate are mediated by GABAB receptors. In: Tunnicliff, G., Cash, C.D. (eds.), Gamma-hydroxybutyrate: molecular, functional, and clinical aspects. London and New York: Taylor and Francis, 28-63, 2002.

5. Bettler, B., Personal communication.

6. Borgen, L. et al., Xyrem® (Sodium oxybate): a study of dose proportionality in healthy human subjects, Journal of Clinical Pharmacology, 40: 1053, 2000.

7. Kaufinan, Elaine E. and Nelson, Thomas, An overview of gamma-hydroxybutyrate catabolism: the role of the cytosolic NADP+-dependent oxidoreductase EC1.1.1.19 and of a mitochondrial hydroxyacid-oxoacid transhydrogenase in the initial, rate-limiting step in this pathway, Neurochemical Research, 16(9): 965-974, 1991.

8. Garlick, A. P. et al, Monitoring flux through the oxidative pentose phosphate pathway using [1-14C] gluconate, Planta, 216: 265-272, 2002.

9. King, Michael W., Reactions of the pentose phosphate pathway, http://www.instate.edu/theme/mwking/pentose-phosphate-pathway.html, Assessed date: Apr. 19th, 2003.

10. Cooper, R. A., The pathway for L-gulonate catabolism in Escherichia Coli K-12 and Salmonella Typhimurium LT-2, FEBS Letters, 115(1): 63-67, 1980.

11. Irwing, S., Drug screening and evaluation of new compounds in animals. In: Nodine, J. and Siegler, P. E. (eds.), Animal and clinical pharmacologic techniques in drug evaluation. Chicago: Year Book Medical Publishers, 36-54, 1964.

12. Quang, L. S. et al, Pretreatment of CD-1 mice with 4-methylpyrazole blocks toxicity from the gamma-hydroxybutyrate precursor, 1,4-butanediol, Life Sciences, 71: 771-778, 2002.

13. Gupta, Yogendra K. et al, Transient focal ischemia induces motor deficit but does not impair the cognitive function in middle cerebral artery occlusion model of stroke in rats, Journal of the Neurological Sciences, 203-204: 267-271, 2002.

14. Green, A. R. et al, The interaction of AR-A008055 and its enantiomers with the GABAA receptor complex and their sedative, muscle relaxant and anticonvulsant activity, Neurophannacology, 41: 167-174, 2001.

15. FAO nutrition meetings report series No. 40A,B,C WHO/Food Add./67.29 toxicological evaluation of some antimicrobials, antioxidants, emulsifiers, stabilizers, flour-treatment agents, acids, acids and bases, http://www.inchem.org/documents/jecfa/jecmono/40abcj42.htm, Assessed date: Apr. 19th, 2003.

16. De Vriendt C. A. et al, Development and validation of a high performance liquid chromatographic method for the determination of GHB in rat plasma, Journal of Chromatography B, Biomedical Science and Application, 752(1): 85-90, 2001.

All publications, patents, and patent applications cited herein are incorporated herein by reference. While the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A therapeutic method comprising administering to a mammal an amount of a compound of formula (I) 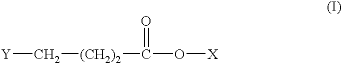
wherein X is H, a pharmaceutically-acceptable cation or (C₁-C₄)alkyl, and Y is OH, (C₁-C₄)alkanoyloxy, (C₁-C₄)alkoxy, phenylacetoxy or benzyloxy, or X and Y together are a single bond, in conjunction with an amount of an inhibitor compound that interferes with the in vivo oxidation of the compound of formula (I) so as to prolong the therapeutic effect of the compound of formula (I).

2. A therapeutic method comprising administering to a mammal an amount of a compound of formula (II) 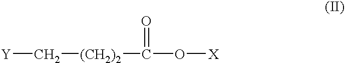
wherein X is H, a pharmaceutically acceptable cation or CO₂X represents an ester linkage to an OH group on an inhibitor compound, and Y is OH, (C₁-C₄)alkanoyloxy, phenylacetoxy or an ester linkage to a carboxylic acid group of an inhibitor compound, wherein the inhibitor compound interferes with the in vivo oxidation of the compound of formula (II) so as to prolong the therapeutic effect of the compound of formula (II).

3. A therapeutic method comprising administering to a mammal an amount of a compound of formula (III): 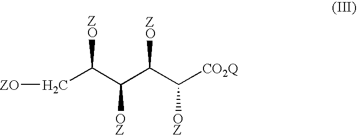
wherein each Z is H or the moiety Y—CH₂(CH₂)₂C(O)—, where at least one Z is Y—CH₂(CH₂)₂C(O)—, wherein Y is OH, (C₁-C₄)alkoxy, (C₁-C₄)alkanoyloxy, phenylacetoxy or benzyloxy, and Q is H, CH₂(CH₂)₂CO₂X or a pharmaceutically acceptable cation, wherein X is H, (C₁-C₄)alkyl or a pharmaceutically acceptable cation.

4. The method of claims 1, 2 or 3 wherein the mammal is a human.

5. The method of claim 4 wherein the human is afflicted with narcolepsy and the therapeutic effect is the reduction of cataplexy.

6. The method of claim 4 wherein the human is afflicted with narcolepsy and the effect is reduction in daytime sleepiness.

7. The method of claim 4 wherein effect is improvement in the quality of sleep.

8. The method of claim 4 wherein the human is an elderly human of >50 years of age.

9. The method of claim 4 wherein the human is afflicted with fibromyalgia or chronic fatigue syndrome and the effect is the alleviation of a symptom of one of fibromyalgia or chronic fatigue syndrome.

10. The method of claims 1, 2 or 3 wherein Y is OH or (C₁-C₄)alkanoyloxy.

11. The method of claims 1, 2 or 3 wherein X is Na⁺.

12. The method of claim 1 wherein Y is OH.

13. The method of claim 3 wherein Q is Na⁺.

14. The method of claim 13 wherein Y is OH.

15. The method of claims 1 or 2 wherein the inhibitor compound is one or more of gluconic acid lactone (GAL), glucuronic acid (GCA), glucuronic acid lactone (GCAL), gulonolactone (GL), gulonic acid (G), or a pharmaceutically-acceptable salt thereof.

16. The method of claims 1 or 2 wherein the inhibitor compound is one or more of phenyl acetic acid, alpha-hydroxyphenyl acetic acid, alpha-ketoglutaric acid, alpha-hydroxyglutaric acid, phenylpyruvic acid, alpha-ketoisocaproic acid, or a pharmaceutically-acceptable salt or ester thereof.

17. The method of claim 1 wherein the compound of formula (I) is administered orally, in combination with a pharmaceutically-acceptable carrier.

18. The method of claim 2 wherein the compound of formula (II) is administered orally, in combination with a pharmaceutically-acceptable carrier.

19. The method of claim 3 wherein the compound of formula (III) is administered orally, in combination with a pharmaceutically-acceptable carrier.

20. The method of claims 17, 18 or 19 wherein the carrier is a liquid.

21. The method of claims 17, 18 or 19 wherein the carrier is a tablet or capsule.

22. The method of claims 17, 18 or 19 wherein a daily dose of about 1-1000 mg/kg of the compound is administered.

23. The method of claims 17, 18 or 19 wherein a daily dose of about 0.5-20 g of the compound is administered.

24. The method of claims 17, 18 or 19 wherein a daily dose of about 1-15 g of the compound is administered.

25. The method of claims 1 or 2 wherein the inhibitor compound is administered orally.

26. The method of claims 1 or 2 wherein the inhibitor compound is administered parenterally.

27. The method of claim 25 wherein the inhibitor compound is administered before administration of the compound of formula (I) or formula (II).

28. The method of claim 26 wherein the inhibitor compound is administered before administration of the compound of formula (I) or formula (II).

29. The method of claim 25 wherein the inhibitor compound is administered at the same time as the compound of formula (I) or formula (II).

30. The method of claim 26 wherein the inhibitor compound is administered at the same time as the compound of formula (I) or formula (II).

31. The method of claim 25 wherein the inhibitor compound is administered in combination with the compound of formula (I) or formula (II).

32. The method of claim 26 wherein the inhibitor compound is administered in combination with the compound of formula (I) or formula (II).

33. The method of claim 1 wherein the inhibitor compound is administered in an amount effective to extend the residence time of a therapeutic level of the compound of formula (I) in the CNS or PNS of said mammal.

34. The method of claim 2 wherein the inhibitor compound is administered in an amount effective to extend the residence time of a therapeutic level of the compound of formula (II) in the CNS or PNS of said mammal.

35. The method of claim 33 or 34 wherein the level is maintained in the brain of the mammal.

36. A composition comprising an amount of a compound of formula (I) or formula (II) in combination with an amount of one or more inhibitor compounds that act so as to interfere with the in vivo oxidation of the compound of formula (I) or formula (II), respectively.

37. A composition comprising an amount of a compound of formula (III) in combination with a pharmaceutically acceptable carrier.

38. The composition of claim 36 wherein the compound of formula (I) is sodium gamma-hydroxybutyrate.

39. The method of claim 1 or 2 wherein the inhibitor compound is present in an amount that reduces the ability of the compound of formula (I) or (II) to cause seizures in said mammal.

40. The method of claim 15 wherein the inhibitor compound is present in an amount effective to reduce the ability of the compound of formula (I) or (II) to cause seizures in said mammal.

41. The method of claim 16 wherein the inhibitor compound is present in an amount effective to reduce the ability of the compound of formula (I) or (II) to cause seizures in a mammal.

42. A compound of formula (II) 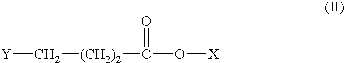
wherein X is H, a pharmaceutically acceptable cation or CO₂X represents an ester linkage to an OH group on an inhibitor compound, and Y is OH, (C₁-C₄)alkanoyloxy, phenylacetoxy or an ester linkage to a carboxylic acid group of an inhibitor compound, wherein the inhibitor compound interferes with the in vivo oxidation of the compound of formula (II) so as to prolong the therapeutic effect of the compound of formula (II).

43. A compound of formula (III): 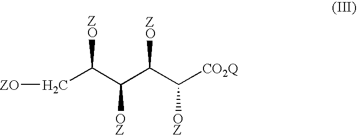
wherein each Z is H or the moiety Y—CH₂(CH₂)₂C(O)—, where at least one Z is Y—CH₂(CH₂)₂C(O)—, wherein Y is OH, (C₁-C₄)alkoxy, (C₁-C₄)alkanoyloxy, phenylacetoxy or benzyloxy, and Q is H, CH₂(CH₂)₂CO₂X or a pharmaceutically acceptable cation, wherein X is H, (C₁-C₄)alkyl or a pharmaceutically acceptable cation.

44. The compound of claims 42 or 43 wherein Y is OH or (C₁-C₄)alkanoyloxy.

45. The compound of claims 42 or 43 wherein X is Na⁺.

46. The compound of claim 42 wherein Y is OH.

47. The compound of claim 43 wherein Q is Na⁺.

48. The compound of claim 47 wherein Y is OH.

49. The compound of claim 42 wherein the inhibitor compound is one or more of gluconic acid lactone (GAL), glucuronic acid (GCA), glucuronic acid lactone (GCAL), gulonolactone (GL), gulonic acid (G), or a pharmaceutically-acceptable salt thereof.

50. The compound of claim 42 wherein the inhibitor compound is one or more of phenyl acetic acid, alpha-hydroxyphenyl acetic acid, alpha-ketoglutaric acid, alpha-hydroxyglutaric acid, phenylpyruvic acid, alpha-ketoisocaproic acid, or a pharmaceutically-acceptable salt or ester thereof.
